(19) United States
(12) Patent Application Publication
(10) Pub. No.: US 2009/0143360 A1
(43) Pub. Date: Jun. 4, 2009

(54) Oxcarbazepine Formulation

(76) Inventors: Muhammed Safadi, Nazareth (IL); Ella Katzir, Haifa (IL); Eleonora Dinisman-Zavulunov, Afula (IL); Nataly Zissman, Nesher (IL); Tamar Blumberg, Kfar-Saba (IL)

Correspondence Address: VENABLE LLP, P.O. BOX 34385, WASHINGTON, DC, 20043-9998, US

(21) Appl. No.: 11/988,433

(22) PCT Filed: Jul. 7, 2006
(86) PCT No.: PCT/US2006/026311

§ 371 Date: Jan. 23, 2008

Related U.S. Application Data
(60) Provisional application No. 60/697,840, filed on Jul. 8, 2005.

(51) Int. Cl. A61K 31/55; A61P 25/08

(52) U.S. Cl. 514/217

(57) ABSTRACT

The present invention relates to novel uncoated, color-stable tablet formulations comprising oxcarbazepine, a disintegrant and iron oxide pigments. The oxcarbazepine of the present invention has a particle size of about 14 to about 30 microns with a maximum residue on a 40 micron sieve from about 10% to about 35%. The present invention further provides for a process of preparing the tablet formulations, and a method of treating mammals in need of oxcarbazepine with the novel formulation.

### BACKGROUND OF THE INVENTION

Oxcarbazepine (10,11-Dihydro-10-oxo-5H-dibenz[b,f]azepine-5-carboxamide) is a 10-ketoanalogue of carbamazepine. It is indicated for use as a monotherapy or as adjunctive therapy for partial seizures in adults and children ages of 4 to 16. Oxcarbazepine is a prodrug that is quickly reduced to a 10-monohydroxy metabolite derivative (MHD) which is the active metabolite. The exact mechanisms by which oxcarbazepine and its active metabolite exert an anticonvulsant effect are unknown. Methods to prepare oxcarbazepine have been described in, for example, U.S. Publication Number 2004-0044200A1.

Oral formulations of oxcarbazepine are commercially available. These formulations are suitable for dosing oxcarbazepine over extended treatment periods to achieve therapeutically effective amounts of active drug. During storage of uncoated white tablets of oxcarbazepine, a non-homogeneous, faintly orange discoloration of the tablet occurs due to the formation of a minor amount (<0.05%) of a pharmacologically harmless oxidation product of the active drug. The oxidation product is known to be caused by an inactive impurity, 10,11-dihydro-5H-dibenzo[b,f]azepine-10,11-dione (U.S. Pat. No. 5,696,782). The discoloration of the tablets is concentration-dependent. In the past, tablet formulations of oxcarbazepine have had iron oxide pigments added to the core of the tablet to mask the faintly orange color. However, the amount of iron oxide in those formulations exceeded the allowable daily amount of iron allowed by the U.S. government. Newer formulations of oxcarbazepine have film-coated the outer layer of the tablets to mask the discoloration. Single and double film coating of tablet cores containing oxcarbazepine have been described in U.S. Pat. Nos. 5,472,714, 5,695,782 and 7,037,525. Film coatings efficiently hide the discoloration of pharmaceutical products, but they also add significantly to the cost, time and the complexity of manufacturing tablets.

Poorly soluble drug substances are often micronized to increase aqueous solubility and bioavailability. Micronization of oxcarbazepine to fine particle size is described in, for example, IE Patent Application No. 904685 and U.S. Pat. No. 7,037,525 (the '525 patent). The '525 patent claims a method of treating seizures by administering a formulation of oxcarbazepine having an improved bioavailability; the oxcarbazepine having a maximum residue on a 40 micron sieve of less than or equal to 5% and/or having a median particle size of approximately 2 microns to 12 microns. Additionally, WO Patent Publication 2006/046105 discloses oxcarbazepine having a median particle size of 14 to 30 microns. U.S. Patent Publication 2006/0111343 discloses an oral dosage form having a median particle size of not less than about 50 microns. Micronization is therefore known to increase the solubility and bioavailability of poorly soluble compounds in general and oxcarbazepine in particular.

The present invention provides for an uncoated oxcarbazepine tablet formulation that is color stabilized by the addition of acceptable amounts of iron oxide pigments. These formulations have good bioavailability, dissolution and efficacy, and are easier and less expensive to produce then the coated tablets. Also described by this invention, is a process to prepare the formulation and a method to use the formulation for treating mammals in need of oxcarbazepine comprising the step of administering a therapeutically effective amount of the pharmaceutical formulation.

### SUMMARY OF THE INVENTION

The presently disclosed and claimed invention is to an uncoated, color-stable tablet pharmaceutical formulation comprising a pharmaceutically effective amount of oxcarbazepine, a disintegrant, and at least one iron oxide pigment. The iron oxide pigment contains about 0.10% (w/w) to about 0.22% (w/w) of elemental iron. Most preferably the formulation contains about 0.11% (w/w) of elemental iron.

The disintegrant may be chosen from the group consisting of polacrilin potassium, starch, pregelatinzed starch, alginic acid, carboxymethylcellulose, sodium cellulose, colloidal silicon dioxide, croscarmellose sodium, magnesium aluminum silicate, methylcellulose, sodium starch glycolate, microcrystalline cellulose, crospovidone, povidone and combinations thereof. The disintegrant may be croscarmaellose sodium, pregelatinized starch, or a combination thereof.

The median particle size of the oxcarbazepine used in the oral formulation may be between from about 14 microns to about 30 microns, or from about 14 microns to about 25 microns. In one embodiment, the median particle size of the oxcarbazepine used to prepare the tablet formulation is from about 14 to about 20 microns, most preferably, the median particle size is from about 16 to about 20 microns. The oxcarbazepine may be micronized to the desired median particle size before formulation into the pharmaceutical composition. The median particle size of the oxcarbazepine used to prepare the formulation has a maximum residue on a 40 um sieve of between about 7% to about 40%, or from about 10% to about 35%, preferably about 14% to about 30%.

The formulation may have oxcarbazepine in an amount up to 600 mg per tablet, more particularly oxcarbazepine is selected from the group consisting of 150 mg, 300 mg or 600 mg per tablet.

The inventive formulation has a coloring agent in the core of the tablet. The coloring agent may be a pigment that may be selected from the group consisting of iron oxide or hydroxides, titanium dioxide, or zinc oxide. The pigment may be iron oxide and may be selected from the group consisting of iron oxide yellow, iron oxide red, iron oxide black and combinations thereof. The iron oxide may be a mixture, and in a particular embodiment the iron oxides may be a mixture of iron oxide yellow and iron oxide red and the ratio may be iron oxide yellow to iron oxide red from about 5 to 1 to about 3 to 0.5

The tablet formulation of the present invention would result in the maximum daily amount of less than about 5 mg of elemental iron based on a 2400 mg per day maximum dose of oxcarbazepine.

The inventive formulation further comprises pharmaceutically acceptable excipients such as binders, lubricants, diluents, disintegrants and mixtures thereof.

The oral formulation of the invention may be used for treating a mammal in need of oxcarbazepine by administering a therapeutically effective amount of the pharmaceutical formulation. It is an object of the invention to administer less than about 5 mg/day of elemental iron to an animal in need of a pharmaceutically acceptable amount of oxcarbazepine.

This invention is directed to an uncoated color stable tablet pharmaceutical formulation comprising:
a. about 69% to about 71% (w/w) oxcarbazepine;
b. about 0.2% to about 2% (w/w) of at least one glidant;
c. about 2% to about 10% (w/w) of at least one binder;
d. about 0.10% to about 0.22% (w/w) elemental iron;
e. about 0.2% to about 2% (w/w) of at least one lubricant; and
f. about 4% to about 30% (w/w) of one or more disintegrants.

In one embodiment, this invention is directed to an uncoated color stable tablet pharmaceutical formulation comprising:
a. about 70% (w/w) oxcarbazepine;
b. about 0.47% (w/w) colloidal silicon dioxide;
c. about 2.3% (w/w) hydroxypropyl methylcellulose;
d. about 0.11% (w/w) iron;
e. about 0.29% (w/w) magnesium stearate;
f. about 13.7% (w/w) microcrystalline cellulose;
g. about 12.9% (w/w) croscarmellose sodium.

In another embodiment this invention comprises an uncoated color stable tablet pharmaceutical formulation comprising:
a. about 71% (w/w) oxcarbazepine;
b. about 1.3% (w/w) colloidal silicon dioxide;
c. about 5.1% (w/w) starch;
d. about 0.11% (w/w) elemental iron;
e. about 0.5% (w/w) magnesium stearate;
f. about 15% (w/w) microcrystalline cellulose;
g. about 5% (w/w) croscarmellose sodium; and
h. about 2% hydroxypropyl methylcellulose.

This invention further comprises an uncoated color stable tablet pharmaceutical formulation comprising:
a. about 69% (w/w) oxcarbazepine;
b. about 0.5% (w/w) colloidal silicon dioxide;
c. about 0.11% (w/w) elemental iron;
d. about 0.29% (w/w) magnesium stearate;
e. about 13.4% (w/w) microcrystalline cellulose;
f. about 13.8% (w/w) croscarmellose sodium; and
g. about 3% hydroxypropyl methylcellulose.
   Also provided by this invention is a process to prepare the uncoated color-stable tablets with a pharmaceutically effective amount of oxcarbazepine, a disintegrant, and an iron oxide pigment. Also provided is a method of treating mammals in need of oxcarbazepine.

### DESCRIPTION OF THE INVENTION

The formulations of the present invention contain a tablet core of oxcarbazepine which has been granulated with color. The color is important during the marketing and shelf-life of pharmaceutical products, as changes to the color may result in an appearance which may erroneously indicate damage to the product and reduce patient compliance. Color stable pharmaceutical products, or products that do not change color over the shelf-life of the product, are desirable.

Iron oxides are known as coloring agents and visually alter the appearance of a pharmaceutical formulation by imparting definite color to the formulation. The iron oxides used in tablet formulation may include iron oxide yellow (Fe₂O₃.H₂O, 97% monohydrate), iron oxide red (Fe₂O₃) and iron oxide black (FeO.Fe₂O₃). Color serves to introduce a uniformity of appearance to the formulation, and as in the present invention, camouflage changes in color that might occur due to minor chemical changes of the active pharmaceutical agent. The use of iron oxide as a colorant has not been thoroughly investigated, but excessive levels of iron may cause liver and kidney damage, possible convulsions and death (Goodman and Gilman, “The Pharmacological Basis of Therapeutics”, 9th edition, 1996 page 1324). Iron oxide as a coloring agent for oral pharmaceuticals has therefore been limited by The United States Food and Drug Administration to a maximum ingestion of 5 mg of elemental iron per day. The amount of iron in pharmaceutical preparations is calculated in terms of the elemental iron to be administered rather than in terms of the iron compound, as it is the elemental iron that is considered to be toxic. By way of this standardization, the amount of any iron preparation can be calculated on the basis of its known molecular weight. For maximum color effect, we have found that a ratio of iron oxide yellow to iron oxide red formed a tablet that had a light peach color that was maintained for six months storage under accelerated conditions (40° C., 75% relative humidity). The ratio of iron oxide yellow to iron oxide red between about 5:1 (yellow:red) to about 3:0.5 (yellow:red) gave a pleasing, stable color. Preferably, the elemental iron oxide of the present formulation is between about 0.10% to about 0.22% (w/w), more preferably between about 0.11% to about 0.2% (w/w), most preferably 0.11% (w/w), of the total composition. This amount of iron allows for a color stable, uncoated product. The term uncoated is meant to denote that no steps were taken in the manufacture of the tablet to deposit an external layer around the tablet core of the invention. The coloring agents of the present invention are granulated into the tablet core.

While oxcarbazepine may be formulated into granules, capsules or other solid pharmaceutical compositions, the tablet form is preferred. Specific examples of the delivery system of the invention are tablets, tablets which disintegrate into granules, or any other means which allow for tablets for oral administration. (U.S. Pat. No. 6,296,873)

The formulations of the invention are prepared by procedures known in the art, such as for example, by dry mixing and/or wet granulation of the pharmaceutical and inactive ingredients. In one method, for example, the tablet is prepared by wet granulation in the presence of water as a granulating fluid. In an alternative method an organic solvent, such as isopropyl alcohol, ethanol, and the like, may be employed with or without water. In the method of the invention, wet milling of the granulate is followed by drying, and further milling. The iron oxide of the inventive formulation is added at both the granulation and dry powder blending steps of the process to form the core of the tablet.

Oxcarbazepine has very low solubility in water, and thus a slow dissolution in aqueous medium. Low aqueous solubility is a deterrent to bioavailability. In solid dosage forms such as tablets, when the dissolution rate is slower than the absorption rate, dissolution rate can become the rate-limiting step in drug absorption. The absorption can be controlled by manipulating the formulation by decreasing the particle size of the compound, thus increasing the surface area of the drug, which allows for better dissolution and increased bioavailability. The particle size may be reduced by any method known to reduce particle size, for example, micronizing the active pharmaceutical ingredients. Particle size distribution may be controlled by pressure and feed rate and other variables known to one skilled in the art. (Remington: The Science and Practice of Pharmacy, 20th Edition. Baltimore, Md.: Lippincoft Williams & Wilkins, 2000).

Fluid milling may be used to micronize the active pharmaceutical ingredients, giving a fine powder having a median particle size of 2 to 30 microns. Decreasing the particle size to fine particles controls the release kinetics of the drug and enhances its solubility. Oxcarbazepine having a fine particle size distribution results in good dissolution characteristics and bioavailability. Fine particle size may be defined as particles that have a median distribution of between about 14 microns to about 30 microns, in particular from about 14 microns to about 20 microns, or between 16 to 20 microns. The median particle size of the oxcarbazpine used to prepare the formulation has a maximum residue on a 40 micron sieve of between about 7% to about 40%, more preferably about 20%.

Appropriate excipients for use in the formulation of oral dosage forms will be known to those skilled in the art (“Handbook of Pharmaceutical Excipients” ed. Kibbe, 3rd edition, 2000). Among the excipients useful for the present formulation are binders, glidants, lubricants and disintegrants. For the formation of tablets, binders such as acacia, alginic acid, carboxymethylcellulose, cellulose, dextrin, gelatin, glucose, guar gum, hydroxypropylmethylcellulose, magnesium aluminum silicate, maltodexterin, methylcellulose, polyethylene oxide, polymethacrylates, povidone and starch may be used in the present formulation.

Lubricants are required during manufacture of a tablet to keep the raw ingredient blend from sticking to the equipment. Lubricants improve the flow of powder mixes through the presses, and they help the finished tablets release from the equipment with a minimum of friction and breakage. Suitable lubricants for use in the present invention include calcium stearate, magnesium oxide, hydrogenated vegetable oil, mineral oil, canola oil, poloxamer, polyethylene glycol, polyvinyl alcohol, sodium benzoate, sodium lauryl sulfate, sodium stearyl fumarate, stearic acid, talc, zinc stearate magnesium stearate and mixtures thereof. Magnesium stearate provided excellent lubricant properties for the present tablet formulation.

Disintegration of the pharmaceutical dosage form increases the surface area of the active agent, and increases the bioavailability of the drug. Disintegrants, such as alginic acid, carboxymethylcellulose, cellulose, colloidal silicon dioxide, croscarmellose sodium, crospovidone, magnesium aluminum silicate, methylcellulose, microcrystalline cellulose, povidone aid in the dissolution of a drug tablet in vivo. Super disintegrants are useful in the present formulations. Sodium starch glycolate (Explotab®), croscarmellose sodium (AC-Di-Sol®) and crospovidone are examples of super disintegrants useful in the present invention. In particular, a combination of super disintegrants and one or more disintegrant is preferred. In one embodiment of the present invention a combination of microcrystalline cellulose and croscarmellose sodium is used.

Oxcarbazepine treatment is typically initiated with a twice daily dose of 300 mg per day, to a maximum of 600 mg/day. Formulations of 150 mg, 300 mg and 600 mg of oxcarbazepine are prepared in tablets or other pharmaceutical compositions. If clinically indicated, the dose may be increased at approximately weekly intervals. Side effects, such as allergic reactions, double vision, and increased seizure activity limit the maximum tolerable daily dosage to 2400 mg/day.

The daily dose of oxcarbazepine may be formulated in a single tablet or more than one tablet, depending on the daily dose of oxcarbazepine and the number of times per day the formulation is to be administered. The amount of oxcarbazepine in the formulation varies depending on the desired dose for efficient drug delivery. The actual drug amount is determined according to intended medical use by techniques known in the art. The pharmaceutical dosage formulated according to the invention may be administered one or more times per day. The amount of oxcarbazepine in the formulation varies as desired for efficient delivery, and is dependent on the patient's age, weight, sex, disease and any other medical criteria, and according to intended medical use by techniques known in the art. Oxcarbazepine formulated according to the invention is designed to be delivered once, twice or up to four times a day, depending on the desired dose.

The invention is illustrated, but in no way limited, by the following examples.

### EXAMPLE 1

TABLE 1
Oxcarbazepine Tablet Composition - Formula A

| Ingredient | mg | mg | mg | % (w/w) |
|---|---|---|---|---|
| Oxcarbazepine | 600 | 300 | 150 | 70 |
| Colloidal Silicon Dioxide | 4 | 2 | 1 | 0.47 |
| Hydroxypropyl | 20 | 10 | 5 | 2.3 |
| Methylcellulose | | | | |
| Elemental Iron | 0.962 | 0.477 | 0.239 | 0.11 |
| Magnesium Stearate | 2.5 | 1.25 | 0.625 | 0.29 |
| Microcrystalline Cellulose | 117 | 58.5 | 29.25 | 13.7 |
| Croscarmellose Sodium | 110 | 55 | 27.5 | 12.9 |

TABLE 2
600 mg dose Oxcarbazepine Tablets

| Formula A | Formula B | Formula C | |
|---|---|---|---|
| 600 | 600 | 600 | Oxcarbazepine |
| 4 | 11 | 4 | Colloidal Silicon Dioxide |
| 117 | 125 | 117 | Microcrystalline Cellulose |
| 110 | 40 | 120 | Croscarmellose Sodium |
| 20 | 17 | 26 | Hydroxypropyl Methylcellulose |
| 0.962 | 0.962 | 0.962 | Elemental Iron |
| 2.5 | 4 | 2.5 | Magnesium Stearate |
| | 43 | | Pregelatinized starch NF/EP |
| | | | (Starch 1500) |

### EXAMPLE 2

The following process was used to form representative oxcarbazepine tablets. Oxcarbazepine (Taro Pharmaceuticals Inc., Haifa, Israel) and microcrystaline cellulose (AVICEL PH 102®, FMC, Cork, Ireland), hydroxypropyl methylcellulose (Pharmacoate 603®, Shinetsu, Tokyo, Japan), croscarmellose sodium (AC-DI-SOL®, Cork, Ireland), were added to a high speed granulator (Collette 75-liter high shear granulator, Model Gral-75 (GEA, Belgium). Collodial silicone dioxide (CAB-O-SIL® M-5P, Cabot Eastech Chemical Inc. Philadelphia, U.S.A) iron oxide yellow and iron oxide red (Warner Sensient Colors UK, LTD., Kings Lynn, Norfolk, England), were passed through a cone mill (Comil) equipped with a 045R screen. The excipients were then added to the high shear granulator. Water was added at a flow rate of 1050 grams/minute, followed by mixing. The wet granulate was then passed through a Comil 197 with a screen size of 250Q, impelled by 1400 RPM (Quadro, Waterloo, Canada). The wet granulate was dried using a fluid bed dryer (Glatt 60/90, Glatt, Germany). The dried granulate was then passed through the Comil 197, Screen 045R, impelled at a speed of 1400 RPM. The dried granulate was transferred to a 930 L V-Blender. Microcrystalline cellulose iron oxide yellow, iron oxide red, croscarmellose sodium (AC-DI-SOL®) and colloidal silicone dioxide (CAB-O-SIL® M-5P) were then added to the blender and mixed for an additional 15 minutes. Magnesium stearate (Merck, Darmstadt, Germany) was screened through a 60 mesh sieve and added to the blender for an additional 5 minutes. The mixture was then compressed in a Fette tabletting machine (Fette, Germany) to form tablets.

### EXAMPLE 3

The following table calculates total amount of elemental iron in the tablets of the present invention, and the total amount of oxcarbazepine in a 2400 mg/day dose of oxcarbazepine.

TABLE 3
Iron (mg) in Oxcarbazepine Tablets, and in 2400 mg/day dose.

| | Iron Oxide Yellow | | Iron Oxide Red | | Total | Number of | Total mg |
|---|---|---|---|---|---|---|---|
| | (mg/tab) | | (mg/tab) | | Elemental | Tablets for | elemental iron/ |
| Oxcarbazepine | Total | Elemental | Total | Elemental | Iron | Max. Daily | day in Max dose |
| (mg/tablet) | Fe₂O₃•H₂O | Iron | Fe₂O₃ | Iron | (mg/tab) | Dose | of oxcarbazepine* |
| 150 | 0.3125 | 0.196 | 0.0625 | 0.043 | 0.239 | 16 | 3.824 |
| 300 | 0.625 | 0.393 | 0.125 | 0.087 | 0.48 | 8 | 3.84 |
| 600 | 1.25 | 0.787 | 0.25 | 0.175 | 0.962 | 4 | 3.848 |

### EXAMPLE 4

Stability studies of the Oxcarbazepine tablets were performed on various batches of different strength tablets. Samples were placed under 3 different environmental conditions:
1) accelerated stability conditions of 40±2° C./75±5% relative humidity;
2) long-term conditions of 25±2° C./60±5% relative humidity; and
3) intermediate conditions 30±2° C./60±5% relative humidity.
   Results were obtained for 6 months under accelerated conditions and 24 months under long-term conditions.

The following specifications were analyzed for stability:
a. appearance as pale peach, ovaloid, slightly biconvex tablet;
b. assay for oxcarbazepine, carbamazepine, 10-methoxy carbamazepine, and 10-oxo-10,11-dihydro-5H-dibenz(b,f)azepine using a HPLC and UV detector and chromatographic methods well known to those skilled in the art.
c. not less than 70% of the labeled amount of oxcarbazepine dissolved in 30 minutes, and not less than 80% of the labeled amount of oxcarbazepine dissolved in 60 minutes when tested under standard dissolution test conditions.

### Results:

Appearance: The appearance of Oxcarbazepine tablets remained as pale peach, ovaloid, slightly biconvex tablet for 6 months under accelerated conditions and for 24 months under long-term conditions. The tablets were color stable.

Dissolution: The dissolution of Oxcarbazepine tablets remained within specifications for 6 months under accelerated conditions and for 24 months under long-term conditions for both the 30 minutes and 60 minutes of dissolution.

Related Compounds: The levels of individual and total related compounds of Oxcarbazepine tablets remained well below the specification limits for 6 months under accelerated conditions and for 24 months under long-term conditions. The amount of the 10-oxo-10,11-dihydro-5H-dibenz(b,f)azepine remained below the lower limit of quantification during the 24 months stability testing.

Assay: No significant change was observed in the assay of Oxcarbazepine tablets as a function of time for 6 months under accelerated and for 24 months long-term conditions. The assay remained within specifications for all types of packages.

In describing embodiments of the present invention, specific terminology is employed for the sake of clarity. However, the invention is not intended to be limited to the specific terminology so selected. It is to be understood that each specific element includes all technical equivalents, which operate in a similar manner to accomplish a similar purpose. The above-described embodiments of the invention may be modified or varied, and elements added or omitted, without departing from the invention, as appreciated by those skilled in the art in light of the above teachings. Each reference cited herein is incorporated by reference as if each were individually incorporated by reference.

1. An uncoated, color stable pharmaceutical formulation comprising a pharmaceutically effective amount of oxcarbazepine, a disintegrant, and an iron oxide pigment wherein the elemental iron of the iron oxide pigment is between about 0.10% (w/w) to about 0.22% (w/w) of the total weight of the tablet.

2. The formulation of claim 1, wherein the disintegrant is chosen from the group consisting of alginic acid, carboxymethylcellulose, cellulose, colloidal silicon dioxide, croscarmellose sodium, starch, pregelatinized starch, sodium starch glycolate, polacrilin potassium, crospovidone, magnesium aluminum silicate, methylcellulose, microcrystalline cellulose, povidone and combinations thereof.

3. The formulation of claim 2, wherein at least one of the disintegrants is a super disintegrant.

4. The formulation of claim 3 wherein the super disintegrant is croscarmellose sodium, pregelatinized starch, or a combination thereof.

5. The formulation of claim 1, wherein the median particle size of the oxcarbazepine is between from about 14 microns to about 30 microns.

6. The formulation of claim 5, wherein the median particle size of the oxcarbazepine is between from about 14 microns to about 20 microns.

7. The formulation of claim 5, wherein the median particle size of the oxcarbazepine is between from about 16 microns to about 20 microns.

8. The formulation of claim 5, wherein the median particle size of the oxcarbazepine has a maximum residue on a 40 micron sieve of from about 14% to about 30%.

9. The formulation of claim 5, wherein the median particle size of the oxcarbazepine has a maximum residue on a 40 micron sieve of about 20%.

10. The formulation of claim 1, wherein the iron oxide is selected from the group consisting of iron oxide yellow, iron oxide red, iron oxide black and combinations thereof.

11. The formulation of claim 10, wherein the iron oxide is a mixture of iron oxide yellow and iron oxide red.

12. The formulation of claim 10, wherein the ratio of the iron oxide yellow to iron oxide red is from about 5 to 1 to about 3 to 0.5.

13. The formulation of claim 1 wherein the amount of iron in the iron oxide is 0.11% (w/w) of the total weight of the tablet.

14. The formulation of claim 1 further comprising pharmaceutically acceptable excipients.

15. The formulation of claim 14 wherein the excipients are selected from the group consisting of binders, lubricants, diluents, glidants, disintegrants and mixtures thereof.

16. The formulation of claim 15 wherein the lubricants are selected from the group consisting of calcium stearate, magnesium oxide, hydrogenated vegetable oil, mineral oil, canola oil, magnesium oxide, poloxamer, polyethylene glycol, polyvinyl alcohol, sodium benzoate, sodium lauryl sulfate, sodium steararyl fumarate, stearic acid, talc, zinc stearate, magnesium stearate, and mixtures thereof.

17. The formulation of claim 16 wherein the lubricant is selected from the group consisting of hydroxypropyl methylcellulose, magnesium stearate, microcrystalline cellulose, colloidal silicon dioxide, pregelatinized starch, starch, croscarmellose sodium and mixtures thereof.

18. The formulation of claim 1 comprising:
a. about 69% to about 71% (w/w) oxcarbazepine;
b. about 0.2% to about 2% (w/w) of at least one glidant;
c. about 2% to about 10% (w/w) of at least one binder;
d. about 0.10% to about 0.22% (w/w) elemental iron;
e. about 0.2% to about 2% (w/w) of at least one lubricant; and
f. about 4% to about 30% (w/w) of one or more disintegrants.

19. The formulation of claim 1 comprising:
a. about 70% (w/w) oxcarbazepine;
b. about 0.47% (w/w) colloidal silicon dioxide;
c. about 2.3% (w/w) hydroxypropyl methylcellulose;
d. about 0.11% (w/w) iron;
e. about 0.29% (w/w) magnesium stearate;
f. about 13.7% (w/w) microcrystalline cellulose;
g. about 12.9% (w/w) croscarmellose sodium.

20. The formulation of claim 1 comprising:
a. about 71% (w/w) oxcarbazepine;
b. about 1.3% (w/w) colloidal silicon dioxide;
c. about 5.1% (w/w) starch;
d. about 0.11% (w/w) elemental iron;
e. about 0.5% (w/w) magnesium stearate;
f. about 15% (w/w) microcrystalline cellulose;
g. about 5% (w/w) croscarmellose sodium; and
h. about 2% hydroxypropyl methylcellulose.

21. The formulation of claim 1 comprising:
a. about 69% (w/w) oxcarbazepine;
b. about 0.5% (w/w) colloidal silicon dioxide;
c. about 0.11% (w/w) elemental iron;
d. about 0.29% (w/w) magnesium stearate;
e. about 13.4% (w/w) microcrystalline cellulose;
f. about 13.8% (w/w) croscarmellose sodium; and
g. about 3% hydroxypropyl methylcellulose.

22. A method for treating a mammal in need of oxcarbazepine comprising the step of administering a therapeutically effective amount of the pharmaceutical formulation of claim 1.

23. The method of claim 22, wherein the mammal is administered less than about 5mg/day of iron.

24. A process to prepare the formulation of claim 1 comprising the steps of:
a. adding oxcarbazepine, iron oxides to a high shear granulator;
b. adding water to the high shear granulator and mixing to form a wet granulate;
c. passing the wet granulate through a mill;
d. drying the milled wet granulate in a fluid bed dryer;
e. blending the dried granulate with iron oxides, and other pharmaceutically acceptable excipients; and
f. compressing the blend of step (e) to form a tablet.
